(19) United States
(12) Patent Application Publication
(10) Pub. No.: US 2019/0040392 A1
(43) Pub. Date: Feb. 7, 2019

(54) TARGETED MRNA FOR IN VIVO APPLICATION

(71) Applicant: Eberhard Karls Universitaet Tuebingen Medizinische Fakultaet, Tuebingen (DE)

(72) Inventors: Michael Kormann, Weil im Schönbuch (DE); Christian Martin Seitz, Tuebingen (DE); Patrick Schlegel, Rottenburg (DE)

(21) Appl. No.: 16/165,598

(22) Filed: Oct. 19, 2018

Related U.S. Application Data
(63) Continuation of application No. PCT/EP2017/059540, filed on Apr. 21, 2017.

(30) Foreign Application Priority Data
Apr. 21, 2016 (EP) 16166310.9

(51) Int. Cl. C12N 15/115; A61K 31/7105; A61K 47/54; A61K 48/00

(52) U.S. Cl. CPC C12N 15/115; A61K 31/7105; A61K 48/005; A61K 48/0016; A61K 47/549

(57) ABSTRACT

A medicament can include a product for in vivo expression of a protein in a living being. The product can include a first entity, which includes a nucleic acid encoding an intracellularly expressible protein, and an associated second entity configured for specific binding to a cellular structure of the living being. One example of the product is a nucleotide-modified mRNA, in which includes a first ribonucleotide sequence encoding the intracellularly expressible protein, and a second ribonucleotide sequence encoding an aptamer configured for specific binding to the cellular structure of the living being.

### CROSS-REFERENCE TO RELATED APPLICATIONS

This application is a continuation of copending international patent application PCT/EP2017/059540, filed on 21 Apr. 2017 and designating the U.S., which international patent application has been published in English, and claims priority from European patent application EP 16 166 310.9, filed on 21 Apr. 2016. The entire contents of these prior applications are incorporated herein by reference.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 29257130_1.TXT, the date of creation of the ASCII text file is Oct. 19, 2018, and the size of the ASCII text file is 7.01 KB.

### FIELD

The present invention relates to a product for an in vivo expression of a protein in a living being, a medicament comprising said product, a method for the treatment of a disease, and to a nucleotide-modified mRNA for an in vivo expression of a protein in a living being.

### BACKGROUND

Numerous genetic diseases are driven by the presence of dysfunctional or insufficiently expressed proteins. To this end, therapeutic approaches have aimed at delivering functionally active protein or its genetically encoded precursors, the corresponding DNA or messenger RNA (mRNA), into patients' cells. In theory, these methods allow for the expression of functionally active protein, leading to the correction of disease. Unfortunately, in practice, many obstacles remain to achieve a safe, long-term protein expression in a clinical setting.

Of these approaches, protein delivery is often hampered by protein metabolism or difficulty incorporating natural protein modifications, while gene delivery using plasmid DNA (pDNA) is commonly limited by CpG motifs that induce strong immune responses through innate immune receptors such as Toll-like receptor 9 (TLR9) and poor transfection efficiency in non- or slowly-dividing mammalian cells. Additionally, the use of viral vectors for gene therapy approaches has been threatened by the risk of insertional mutagenesis following random integration events that may occur within an oncogene or tumor suppressor. The development of immune responses against the viral capsid may also occur, which can prevent the possibility of vector re-administration.

For this reason, researchers have begun to explore the potential for mRNA to serve as an efficient, safe, non-integrating delivery vehicle for in vivo gene therapy. By micking endogenous mRNA modifications, the immune-stimulatory effects of in vitro transcribed mRNA can be prevented thus paving the way for transcript supplementation therapy as an efficient alternative to viral vectors. In 2011, it was demonstrated that in vivo delivery of nucleotide modified mRNA could achieve therapeutic levels of protein expression in murine lung; see Kormann, M. S., et al., Expression of therapeutic proteins after delivery of chemically modified mRNA in mice. Nat Biotechnol, 2011. 29(2): p. 154-7 and Mclvor, R. S., Therapeutic delivery of mRNA: the medium is the message. Molecular therapy: The Journal of the American Society of Gene Therapy, 2011, 19(5): p. 822-3. Unlike gene therapy with most viral vectors, the use of modified mRNA allowed for multiple administrations of therapeutic transcript, while providing a safer alternative that prevented immune activation and eliminated the possibility of genomic integration.

Uchida et al., Systemic delivery of messenger RNA for the treatment of pancreatic cancer using polyplex nanomicelles with a cholesterol moiety. Biomaterials 2016, 82: p. 221-8, describe the in vivo administration of mRNA encoding an anti-angiogenic protein (sFlt-1) packed in nanomicelles into mice suffering from a pancreatic tumor. The authors describe an inhibitory effect on the tumor growth.

However, so far the in vivo administration or delivery of mRNA is characterized by a low target-finding capacity. The administered mRNA is, in principle, spread all over the body or accumulated in a large number of tissues or the cardiovascular system, and only a low amount reaches the target cells. Therefore, the occurrence of side effects and systemic toxicity poses a severe problem in this approach.

A concept where an unspecific distribution of therapeutic or recombinant nucleic acid in a patient's body can be controlled to a better degree is the so-called adoptive cell transfer (ACT). ACT is the transfer of previously autologous or non-autologous withdrawn cells, most commonly derived from the immune system, into a patient, with the goal of transferring improved immune functionality and characteristics along with the cells back to the patient. The cells can be treated ex vivo with the recombinant nucleic acid or a vector encoding for a desired functionality by means of genetic engineering, or an immunogen or drug, thereby effecting an alteration or reprogramming of the withdrawn cells, before re-administering them into the patient. As of 2015 ACT had expanded to treat cervical cancer, lymphoma, leukemia, bile duct cancer and neuroblastoma; see Rosenberg et al., Adoptive cell transfer as personalize immunotherapy for human cancer. Science 348 (6230): p. 62-68, and in 2016, lung cancer, breast cancer, sarcoma and melanoma. In 2016 CD19-specific chimeric antigen receptor (CAR)-modified T cells were used to treat patients with relapsed and refractory CD19+ B cell malignancies, including B cell acute lymphoblastic leukemia (B-ALL) harboring rearrangement of the mixed lineage leukemia (MLL) gene with CD19 CAR-T cells, see Gardner et al., Acquisition of a CD19 negative myeloid phenotype allows immune escape of MLL-rearranged B-ALL from CD19 CAR-T cell therapy. Blood: blood-2015-08-665547.

However, the ACT technology is very complex and expensive. The withdrawn cells are to be individualized and prepared for each and every patient separately. Therefore the ACT technology can only be provided by highly specialized clinics resulting in the exclusion of a large number of potentially needy patients.

### SUMMARY

Against this background the object underlying the invention is to provide a new product allowing the targeted expression of a protein in a living being, by means of which the disadvantages of the approaches and technologies in the prior art can be avoided or reduced, respectively.

This problem is solved by the provision of a product for an in vivo expression of a protein in a living being, comprising:
a first entity comprising a first nucleic acid encoding an intracellularly expressible protein, and, associated therewith,
a second entity configured for a specific binding to a cellular structure of said living being.

The inventors have surprisingly realized that with this product that combines the first entity which ensures the in vivo expression of any desired protein in a living being, and the second entity providing for the target-finding capacity, all features are embodied which are essential to effect a therapeutically useful phenomena in the living being.

According to the invention “product” refers to a biologically active molecule such as a nucleic acid, a peptide, a protein, a chemical structure, a macromolecule or a mixture thereof.

According to the invention a “protein” refers to a biomolecule or macromolecule consisting of one or more long chains of amino acid residues and explicitly includes a peptide and polypeptide.

The “first entity” and the “second entity” are functionally distinct units, not necessarily but possibly also structurally distinct units of the product according to the invention. Both entities are associated with each other, either by non-covalent or covalent binding, e.g. phosphodiester bonds in cases where not only the first entity comprises a (first) nucleic acid but also the second entity comprises a (second) nucleic acid molecule(s), e.g. in cases where both entities are realized by mRNA molecules. An example for a non-covalent association or binding is the nucleic acid hybridization again in cases where not only the first entity comprises a (first) nucleic acid but also the second entity comprises a (second) nucleic acid molecule(s).

The first and second entities may each be embodied by a single molecule, e.g. a single nucleic acid molecule. However, each of the entities may also be represented by two, three, four, five or more molecules.

While the “first entity” comprises a conventional nucleotide coding sequence the “second entity” may be a capture or binding molecule being specific and selective for the cellular structure and may be embodied, e.g., by an aptamer, an immunoglobulin, a mono- or polyclonal antibody etc. In case of the second entity being an aptamer the latter may comprise a second nucleotide sequence coding for a spatial motive allowing the specific binding to the cellular structure of said living being.

An “intracellularly expressible protein” as encoded by the first entity or the first nucleic acid, respectively, is a protein that, when the first entity or first nucleic acid is internalized into a cell of the living being, it is converted into a protein, e.g. by using the cellular translation machinery.

A “cellular structure” of said living being is a biological formation present in the living being, which the second entity can specifically bind to, such as an antigen or a surface receptor of a cell, e.g. an immune cell, including a T cell.

A “specific binding” refers to a selective binding of the second entity to the cellular structure as opposed to a non-specific, non-selective binding. An example for a specific binding is the binding of an aptamer or an antibody to their specific targets.

According to the invention a “living being” includes animals of all kinds and a human being.

The product according to the invention is configured for an expression “in vivo”, i.e. for an administration or delivery into the living being and the expression in the living being's cell inside of the organism.

In contrast to the therapeutic approaches used so far in the state of the art based on ex vivo modifications of cells of the living being, such as the ATC technology, the product according to the invention can be produced “off the shelf” and by means of simple injection can be transferred into the patient. Moreover, the invention uses the living being's entire cell reservoir as the potential expression machinery but not only a few number of isolated and ex vivo prepared cells which then need to be re-infused. This results in a significantly improved expression or presence of the transferred protein coding sequence or transgen, respectively.

The second entity of the product according to the invention allows the targeted addressing of any cellular structures, such as specific cell sub-populations, like immune cells including T cells, e.g. T cells with a stem-cell like phenotype.

Another advantage of the product according to the invention is the transient expression of the protein encoded by the first nucleic acid. This allows the expression and, in turn, the biological activity of said protein being limited in time or controlled in time by an repetitive administration of the product according to the invention. Side effects are herewith reduced resulting in an improved safety profile.

The problem underlying the invention is herewith completely solved.

In an embodiment of the invention said first nucleic acid is a messenger ribonucleic acid (mRNA), preferably a nucleotide-modified mRNA.

This measure has the advantage that the first entity is provided in a form allowing the direct transfer or translation of the encoded information into the desired protein by using the cellular protein synthesis machinery.

According to the invention “nucleotide-modified messenger RNA” refers to such an mRNA, where a part of the nucleotides, or nucleosides or nucleobases is modified or changed, respectively. In this respect the terms “nucleotides” and “nucleosides” are used interchangeably. Preferably it is referred to a chemical modification. This modification has the result that the mRNA is more stable and has less immunogenicity. Nucleotide-modified messenger RNA is generally known in the prior art, cf. for example WO 2011/012316. The content of the before-mentioned publication is incorporated herein by reference. Examples for chemically-modified nucleotides or nucleosides are pseudouridine (Ψ), N1-methylpseudouridine (me1-Ψ), 5-methoxyuridine (5-moU), 5-methylcytidine (m5C), N6-methyladenosine (m6A), 5-methyluridine (m5U) or 2-thiouridine (s2U).

According to an embodiment up to including approx. 100% of the uridine nucleotides and/or up to including approx. 100% of the cytidine nucleotides, preferably up to including approx. 70% of the uridine nucleotides and/or up to including approx. 70% of the cytidine nucleotides, further preferably up to including approx. 50% of the uridine nucleotides and/or up to including approx. 50% of the cytidine nucleotides, further preferably up to including approx. 25% of the uridine nucleotides and/or up to including approx. 25% of the cytidine nucleotides, and further preferably approx. 10% of the uridine nucleotides and/or approx. 10% of the cytidine nucleotides of the mRNA are modified, further preferably by exchanging uridine for 2-thiouridine (s2U) and/or pseudouridine (LP) and/or by exchanging cytidine for 5-methylcytidine (m5C).

A depletion of uridine (U) and the use of chemically-modified nucleotides may result in such a first nucleic acid or mRNA which is not immunogenic without the need of HPLC purification. In a large scale waiving of HPLC purification significantly reduces the costs of the product according to the invention.

Further examples of chemically-modified nucleotides or nucleosides include thiouridine, N1-methylpseudouridine, 5-hydroxymethylcytidine, 5-hydroxymethylcytidine, 5-hydroxymethyluridine, 5-methylcytidine, 5-methoxyuridine, 5-methoxycytidine, 5-carboxymethylesteruridine, 5-formylcytidine, 5-carboxycytidine, 5-hydroxycytidine, thienoguanosine, 5-formyluridine. Each of theses chemically-modified nucleotides or nucleosides are, independently from and/or in combination with each other, suitable to replace its non-modified counterpart by up to approx. 10%, preferably up to approx. 25%, further preferably up to approx. 50%, further preferably up to approx. 70%, further preferably up to approx. 100%. Preferred combinations are 5-methylcytidine/thiouridine; 5-hydroxymethylcytidine/5-hydroxymethyluridine; 5-methylcytidine/pseudouridine; 5-methoxyuridine/5-methyluridine; 5-hydroxymethylcytidine/N1-methylpseudouridine; 5-methylcytidine/N1-methylpseudouridine; 5-methylcytidine/5-carboxymethylesteruridine; 5-methoxycytidine/N1-methylpseudouridine; 5-hydroxymethylcytidine/5-methoxyuridine; 5-methylcytidine/thienoguanosine; 5-methylcytidine/5-formyluridine.

This measure has the advantage that through the prescribed content of nucleotide modifications an mRNA is provided which is stable in vivo and little to zero immunogenic. Even more, the inventors could surprisingly realize that it is sufficient if only up to including about 10% to approx. 25% of the non-modified nucleotides or nucleosides are replaced by their modified counterparts. The inventors could provide evidence that also such slightly modified mRNA is stable and little to zero immunogenic. Since the nucleotide modification is complex, this has the advantage that the product according to the invention, because of the low concentration of nucleotide modifications and the possible circumvention of HPLC purification, can be produced in a cost-saving manner. Besides of reducing costs, the lowering of the portion of modified nucleotides has also the advantage that the efficiency of the translation is increased. This is because very high portions of specifically modified nucleotides significantly interfere with the translation of the modified mRNA. However, with lower portions an optimum translation can be observed.

In another embodiment of the invention said intracellularly expressible protein is a protein capable of effecting at least one of the following phenomena in said living being in a targeted manner: immune response, cytokine expression, cell death induction, cell death inhibition, transcription factor expression, genetic modification, epigenetic modification.

The person of skill in the art is perfectly aware of the type of expressible protein to be chosen to achieve the intended effect.

“Cell death” is to be understood as encompassing all phenomena resulting in the dying of cells, including apoptosis with triggering of suicide proteases in the caspase cascade.

This measure has the advantage that the product according to the invention initiates biological responses in the living being which trigger a therapeutically useful effect.

In another embodiment of the product according to the invention said intracellularly expressible protein is an antigen-specific receptor.

This measure has the advantage that an antigen specific receptor, in particular if expressed in an immune cell, allows the targeted induction of an immune response specifically directed against the respective antigen. In doing so, the immune cell of the living being is re-programmed to activate the adaptive immune system against the antigen.

In another embodiment of the product according to the invention said antigen-specific receptor is selected from the group consisting of:
T-cell receptor, preferably a tumor antigen-specific T-cell receptor (e.g. WT-1, NY-ESO1, MAGE-A1, MAGE-A2 etc.), a virus antigen specific T-cell receptor, a bacterium antigen-specific T-cell receptor, a fungus antigen-specific T-cell receptor, a protozoan antigen-specific T-cell receptor; or
a chimeric antigen receptor (CAR),
   preferably a CAR targeting a tumor-associated antigen, preferably selected from the group consisting of: HER2/neu, ErbB, EGFR, EG-FRvIII, FGFR3, FGFR4, LI-13R, II-13Rα2, II-11Rα, VEGFR2, ALK, GD2, GD3, mesothelin, Survivin, PMSA, PSCA, CEA, MUC1, GPC3, GPC5, CSPG4, ROR1, FR-α, FR-β, Igk, Lewis^{Y}, Glypican3, EphA2, CAIX, AFP, FAP, c-MET, HLA-DR, CA-125, CS1, BCMA, NKG2D ligands (MICA/MICB), CLL1, TALLA, LGR5, PD-L1, PD-L2, CD10, CD11b, CD14, CD15, CD19, CD20, CD22, CD29, CD30, CD32, CD33, CD34, CD38, CD44, CD44v6, CD44v7/8, CD45, CD47, CD56, CD64, CD66, CD79a, CD79b, CD95, CD99, CD112, CD117, CD123, CD133, CD135, CD138, CD146, CD152, CD157, CD171, CD184, CD200, CD221, CD243, CD262, CD276, CD300f, CD305, CD326, CD338, CD366; or
   a CAR targeting a bacterium specific antigen, and/or
   a CAR targeting a fungus specific antigen, and/or
   a CAR targeting a virus specific antigen, and/or
   a CAR targeting a protozoan specific antigen.

This measure has the advantage that such an antigen-specific receptor is encoded which has the capability to induce an effective and targeted immune response against the respective antigen.

In a further embodiment of the invention said intracellularly expressible protein is
a cell death inducing or inhibiting protein, preferably selected from the group consisting of: a caspase, a second mitochondria-derived activator of caspases (SMAC), a BCL-2 family protein, an inhibitor of apoptosis protein (IAP), a tumor necrosis factor receptor superfamily (TNFRSF) protein, the death-inducing signaling complex, p53, and interferons, or
an immune modulatory protein, preferably selected from the group consisting of: cytokines, chemokines, tumor necrosis factor (TNF) family proteins and colony stimulating factors, or
a gene expression or protein modulating cellular signaling molecule, preferably selected from a group consisting of: kinases, phosphatases, acetyltransferases, deacetylases, methyltransferases, SUMOylating enzymes, and deSUMOylating enzymes, or
a gene sequence modulating molecule, preferably selected from the group consisting of: zinc-finger nucleases, meganucleases, TAL effector nucleases, CRISPR/Cas9 related nucleases, nickases, and FokI based dCas9 nucleases.

This measure allows the targeted treatment of diseases characterized by degenerated cells. Such degenerated cells will be subjected to cell death or apoptosis resulting in its specific killing, however without involving non-degenerated or healthy cells, respectively. Alternatively, cell death can be purposefully inhibited, the immune response or cellular signaling or gene sequence can be modulated in a targeted fashion.

Example of suitable cytokines include interleukins IL-1, -2, -3, -4, -5, -6, -7, -8, -9, -10, -11, -12, -13, -14, -15, -16, -17, -18, -19, -20, -21, -22, -23, -24, -25, -26, -27, -28, -29, -30, -31, -32, -33, -34, -35, and IL-36. Chemokines include CCL1, -2, -3, -4, -5, -6, -7, -8, -9, -10, -11, -12, -13, -14, -15, -16, -17, -18, -19, -20, -21, -22, -23, -24, -25, -26, -27, and CCL28; CXCL1 to CXCL17; XCL1, XCL2; CX3CL1; RANTES; type I interferons; IFN-α, -β, -ε, -κ, -τ, -δ, -ζ, -ω, -v; type II interferon; IFN-γ; type III interferons. Tumor necrosis factor (TNF) family proteins include TNFα, TNFβ, CD40L, CD27L, CD30L, FASL, 4-1BBL, Ox40L, TRAIL. Colony stimulating factors include M-CSF, G-CSF and GM-CSF. Other immune stimulatory or inhibitory proteins can also be chosen such as CD70, CD80, CD86, ICOSL, PD-L1, PD-L2.

In an embodiment the gene expression or protein modulating cellular signaling molecules also include general activators or inhibitors of gene transcription or translation or cell surface and/or intracellular signal transducing receptors.

In another embodiment of the product according to the invention said cellular structure which can be bound by the second entity is a cell surface molecule, preferably a cell surface expressed protein, further preferably a protein characterizing a cell of the human hematopoiesis and/or a cell of the human immune system, i.e. an immune cell.

With this measure the invention takes advantage of such cellular structures which function as biomarkers allowing the specific addressing of individual subpopulations of cells. Such subpopulations expressing the respective cell surface molecule will, when being bound by the second entity, e.g. the aptamer or antibody, internalize the product according to the invention or, at least, the first entity comprising the first nucleotide acid and ensure that the encoded protein will by expressed.

In another embodiment of the invention said cell surface expressed protein recognized and bound by the second entity is a cluster of differentiation (CD) protein or equivalent, preferably said CD protein is selected from the group consisting of: CD4, CD8, CD3, CD10, CD16, CD19, CD20, CD22, CD25, CD28, CD30, CD33, CD34, CD38, CD44, CD44v6, CD44v7/8, CD45, CD45RA, CD45RO, CD56, CD62L, CD95, CD123, CD127, CD133, CD135, CD137, CD138, CD152, CD171, and preferably said equivalent is selected from the group consisting of: CCR4, CCR5, CCR6, CCR7, CXCR3, CXCR4, CXCR5, TCRαβ, TCRγδ, CTLA-4, PD1, TIM3, NKG2D, HER2/neu, ErbB, EGFR, EG-FRvIII, FGFR3, FGFR4, LI-13R, II-13Rα2, II-11Rα, VEGFR2, ALK, GD2, GD3, mesothelin, survivin, PMSA, PSCA, CEA, MUC1, GPC3, GPC5, CSPG4, ROR1, FR-α, FR-β, Igk, Lewis^{Y}, glypican3, EphA2, CAIX, CSPG4, AFP, FAP, c-MET, HLA-DR, CA-125, CS1, BCMA, NKG2D ligands (MICA/MICB), PD1, PD-L1, PD-L2, CLL1, TALLA, LGR5.

This measure makes use of such surface expressed proteins which characterize and identify immune cells, thereby allowing a specific delivery of the product to immune cells.

According to another embodiment of the product of the invention said second entity is an aptamer.

This measure has the advantage that such a binding molecule is used with can be generated against almost any target structure of interest. Further, it can be easily associated with the first entity, e.g. by attaching it to the first entity or first nucleic acid/first nucleotide sequence via the simple concatenation of nucleotides of the first nucleic acid and nucleotides of the aptamer, e.g. via a phosphodiester bonds. This can be easily realized by methods of nucleic acid synthesis. The aptamer can be attached to the 5′ end or the 3′ end of the first nucleic acid or, if more than one aptamer is used, e.g. two aptamers, one can be attached to the 5′ end and another can be attached to the 3′ end. Alternatively, the aptamer can be associated with the first nucleic acid by hybridizing nucleotides of the aptamer to complementary nucleotides of the first nucleic acid.

Therefore, it is an embodiment where said aptamer is an RNA aptamer, i.e. an aptamer consisting of or comprising ribonucleotides.

This measure allows an easy synthesis of the product according to the invention in form of a single ribonucleic acid molecule comprising the first nucleotide sequence encoding the intracellularly expressible protein and a second nucleotide sequence encoding the aptamer.

As a consequence, in another embodiment of the invention said aptamer is connected to said first nucleic acid by the concatenation of nucleotides resulting in a single-stranded nucleic acid molecule, preferably a single-stranded mRNA molecule, or wherein said aptamer is connected to said nucleic acid by the hybridization of complementary bases resulting in a double-stranded nucleic acid molecule, preferably a double-stranded mRNA molecule.

In another embodiment the product according to the invention comprises nanoparticles complexed with said first and/or said second entity and/or comprises liposomes packaging said first and/or said second entity.

As the inventors were able to realize, the complexation of the first and/or second entity with a nanoparticle, e.g. a chitosan-coated PLGA nanoparticle, or the packaging into liposomes significantly increases the degree of internalization of the product according to the invention into the target cells.

Another subject-matter of the invention relates to the product specified above for the treatment a disease, preferably selected from the group consisting of: a tumor and/or oncologic disease, a hematologic disease, an infectious disease, a rheumatologic disease, a genetic/hereditary disease, an autoimmune disease, an allergic disease.

The features, characteristics and advantages of the product according to the invention apply likewise to this subject-matter.

Another subject-matter relates to a medicament comprising the product according to the invention and a pharmaceutically acceptable carrier.

For this purpose, a “pharmaceutically acceptable carrier” is understood to mean any excipient, additive, or vehicle that is typically used in the field of the treatment of the mentioned diseases and which simplifies or enables the administration of the product according to the invention to a living being, and/or improves its stability and/or activity. The pharmaceutical composition can also incorporate binding agents, diluting agents or lubricants. The selection of a pharmaceutical carrier or other additives can be made on the basis of the intended administration route and standard pharmaceutical practice. As pharmaceutical acceptable carrier use can be made of solvents, extenders, or other liquid binding media such as dispersing or suspending agents, surfactant, isotonic agents, spreaders or emulsifiers, preservatives, encapsulating agents, solid binding media, depending upon what is best suited for the respective dose regime and is likewise compatible with the compound according to the invention. An overview of such additional ingredients can be found in, for example, Rowe (Ed.) et al.: Handbook of Pharmaceutical Excipients, 7^{th} edition, 2012, Pharmaceutical Press.

The features, characteristics and advantages of the product according to the invention apply likewise to the medicament according to the invention.

Another subject-matter of the invention relates to a nucleotide-modified mRNA for an in vivo expression of a protein in a living being comprising:
a first ribonucleotide sequence encoding an intracellularly expressible protein, and
a second ribonucleotide sequence encoding an aptamer configured for a specific binding to a cellular structure of said living being.

The features, characteristics and advantages of the product according to the invention apply likewise to the nucleotide-modified mRNA according to the invention and vice versa.

Another subject-matter relates to a method for the treatment of a disease, preferably a tumor and/or oncologic disease, a hematologic disease, an infectious disease, a rheumatologic disease, a genetic/hereditary disease, an autoimmune disease, an allergic disease, comprising the administration of the product according to the invention and/or the medicament according to the invention into a living being.

In a further embodiment of the method according to the invention the product and/or medicament is administered intravenously (i.v.). In embodiments it comprises a single and/or multiple administrations and continuous administration, e.g. via a drip or pump.

The features, characteristics and advantages of the product according to the invention apply likewise to the method according to the invention and vice versa.

It is to be understood that the before-mentioned features and those to be mentioned in the following cannot only be used in the combination indicated in the respective case, but also in other combinations or in an isolated manner without departing from the scope of the invention.

The invention is now further explained by means of embodiments resulting in additional features, characteristics and advantages of the invention. The embodiments are of pure illustrative nature and do not limit the scope or range of the invention.

The features mentioned in the specific embodiments are also features of the invention in general, which are not only applicable in the respective embodiment but also in an isolated manner in the context of any embodiment of the invention.

The invention is also described and explained in further detail by referring to the drawings.

### BRIEF DESCRIPTION OF THE DRAWINGS

FIG. 1 illustrates therapeutic approaches of the state of the art aimed at delivering functionally active protein or its precursors, exemplified by cystic fibrosis transmembrane conductance regulator (CFTR), into patient's cells; Supplementation of a cell with (A) functional CFTR protein, (B) CFTR cDNA, or (C) mRNA transcripts;

FIG. 2 shows the result of an analysis by means of cytometry of blood cells withdrawn from mice after the administration in vivo of nucleotide-modified mRNA encoding red fluorescent reporter protein (RFP) assembled to nanoparticles into mice for the expression of RFP;

FIG. 3 illustrates the in vivo immune reaction represented by IFN-alpha as measured by ELISA, initiated in mice after the administration in vivo of nucleotide-modified mRNA encoding RFP assembled to nanoparticles;

FIG. 4 shows the result of an analysis by means of cytometry of immune (CD4+) cells withdrawn from NOD.Cg-Prkdc^{scid}II2rg^{tm1Wjl}/SzJ (NSG) mice previously transplanted with human peripheral blood mononuclear cells (PBMCs), after the i.v. administration into mice of aptamer-targeted nucleotide-modified mRNA (atmRNA) encoding RFP and an anti-CD4 aptamer assembled to nanoparticles into mice for the expression of RFP;

FIG. 5 illustrates the structure of the an atmRNA consiting of an aptamer (ar) targeting an effector antigen (ea) and a transgen (tg) (e.g. a chimeric antigen receptor) that is encoded by modified mRNA (mr).

FIG. 6 demonstrates in vitro anti-CD19-CAR expression by flow cytometry on human CD4 and CD8 positive T cells as well as CD14 positive monocytes after electroporation peripheral blood mononuclear cells (PBMCs) with anti-CD19-CAR encoding mRNA or incubation of PBMCs with CD4-targeted anti-CD19-CAR encoding atmRNA alone or assembled to nanoparticles.

FIG. 7 shows in vitro lysis of CD19 positive leukemic blasts (cell line Nalm6) by anti-CD19-CAR expressing T cells at different effector to target ratios. Expression was achieved by either electroporation or pre-incubation with CD4-targeted anti-CD19 CAR encoding atmRNA alone or assembled to nanoparticles as demonstrated in FIG. 6.

FIG. 8 shows the result on an analysis by means of cytometry of immune (CD4+) cells withdrawn from mice after the administration in vivo of atmRNA encoding anti-CD19 CAR and an anti-CD4 aptamer assembled to nanoparticles into mice for the expression of anti-CD19 CAR.

FIG. 9 demonstrates the result of an analysis by flow cytometry of human CD4 and CD8 positive T cells withdrawn from mice after transplantation of pre-activated human T cells and administration in vivo of CD4-targeted atmRNA encoding anti-CD19-CAR assembled to nanoparticles into NSG mice for the expression of anti-CD19-CAR.

FIG. 10 shows the result of an analysis of leukemia (cell line Nalm6) infiltration of bone marrow, analyzed by flow cytometry, in NSG mice after treatment with pre-activated human T cells with or without in vivo application of CD4-targeted atmRNA encoding anti-CD19-CAR assembled to nanoparticles.

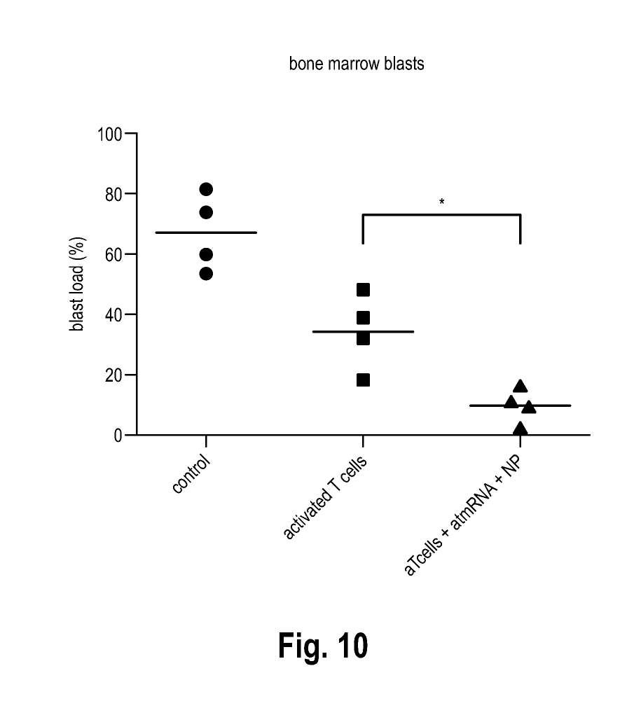

FIG. 11 illustrates the modular design of the product according to the invention.

FIG. 12 illustrates different strategy how immune receptors, expressed by atmRNA, can modulate effector as well as target cell function.

### EXAMPLES

### 1. Methods of the Prior Art

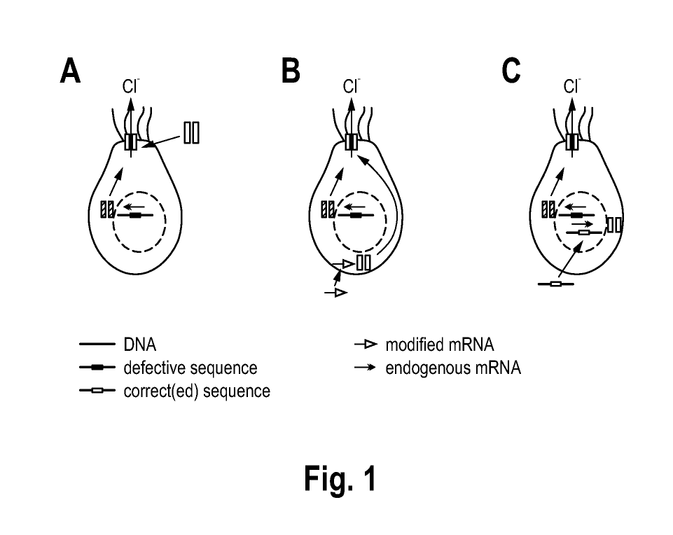

FIG. 1 illustrates methods of the art for restoring functional protein expression in the setting of genetic disease. In this example, a genetic mutation in the cystic fibrosis transmembrane conductance regulator gene, CFTR, leads to faulty expression of the CFTR protein, a chloride ion channel anchored in the plasma membrane. By supplementing the cell with functional CFTR protein (A), CFTR cDNA (B), or mRNA transcripts (C), there is potential to overcome the genetic defects underlying this disease.

In the protein supplementation therapy shown in (A) a correct version of CFTR is transfected or transduced into the respective target cells. The protein delivery is often ineffective and it is difficult to include all natural post-protein modifications. In the transcript supplementation therapy shown in (B) a correct version of CFTR-mRNA is transfected into the respective target cells. Note the mRNA is actively producing CFTR already in the cytoplasm, thereby circumventing the nuclear membrane. In the gene supplementation therapy shown in (C) a correct version of the CFTR gene is transfected or transduced into the respective target cells. Note that the DNA has to enter the nucleus to be transcribed, which is a major barrier in gene therapy. Furthermore, gene delivery using plasmid DNA is commonly limited by CpG motifs that induce strong immune responses through innate immune receptors such as Toll-like receptor 9 (TLR9) and poor transfection efficiency in non- or slowly-dividing mammalian cells. Additionally, the use of viral vectors for gene therapy approaches has been threatened by the risk of insertional mutagenesis following random integration events that may occur within an oncogene or tumor suppressor. The development of immune responses against the viral capsid may also occur, which can prevent the possibility of vector re-administration.

2. Design of an Aptamer Targeted mRNA (atmRNA)

The inventors have developed a nucleotide-modified mRNA that can be delivered intravenously (i.v.) in mice to reprogram T cells, thus targeting a specific antigen. The antigen may be located on any cell of the human body such as tumor cells, on viruses, bacteria or funghi.

The disclosed mRNA consists of a part that encodes for a chimeric antigen receptor (CAR) and it consists—downstream of the CAR encoding part—of a non-protein coding aptamer sequence. The aptamer sequence is able to target e.g. CD4+ T cells.

The DNA sequence encoding the anti-CD4 aptamer is as follows:

(SEQ ID No. 1)
5′-GGGAGACAAGAATAAACGCTCAATGACGTCCTTAGAATTGCGCA
TTCCTCACACAGGATCTTTTCGACAGGAGGCTCACAACAGGC-3′.

In the corresponding mRNA sequence of the anti-CD4 aptamer each “T” (thymine) is replaced by an “U” (uracil).

As the aptamer is critical for the specificity of the mRNA, the inventors call this mRNA “atmRNA” (aptamer targeted mRNA).

3. Functional Tests Demonstrating the Activity of the atmRNA

First, the inventors tested such an mRNA using a red fluorescent reporter protein (RFP) as the encoding part without aptamer sequence. For this experiment RFP DNA was subcloned into the pVAX-A120 vector; see Kormann et al (I.c.). For in vitro transcription (IVT) of chemically modified mRNA the plasmid was linearized with XhoI and transcribed in vitro using the MEGAscript T7 Transcription kit (www.lifetechnologies.com), incorporating 25% 2-thio-UTP and 25% 5-methyl-CTP or 100% PseudoUTP and 100% 5-methyl-CTP (all from www.trilinkbiotech.com). The anti reverse CAP analog (ARCA)-capped synthesized nec-mRNAs were purified using the MEGAclear kit (www.lifetechnologies.com) and analyzed for size on agarose gels and for purity and concentration on a NanoPhotometer (http://www.implen.com),

In the first experiment (modified RFP-mRNA) the inventors assembled the mRNA to nanoparticles (NPs) with Chitosan-coated PLGA using the following protocol: Chitosan (83% deacetylated (Protasan UP CL 113, www.novamatrix.biz)) coated PLGA (poly-d,l-lactide-co-glycolide 75:25 (Resomer RG 752H, www.evonik.de) nanoparticles (short: NPs) were prepared by using emulsion-diffusion-evaporation15 with minor changes. In brief, 100 mg PLGA was dissolved in ethyl acetate and added dropwise to an aqueous 2.5% PVA solution (polyvinyl alcohol, Mowiol 4-88, www.kuraray.eu) containing 15 mg Chitosan. This emulsion was stirred (1.5 h at room temperature) and followed by homogenization at 17,000 r.p.m. for 10 min using a Polytron PT 2500E (www.kinematica.ch). These positively charged NPs were sterile filtered and characterized by Malvern ZetasizerNano ZSP (hydrodynamic diameter: 157.3 Å} 0.87 nm, PDI 0.11, zeta potential +30.8 Å} 0.115 mV). After particle formation they were loaded with mRNA by mixing (weight ratio, 25:1).

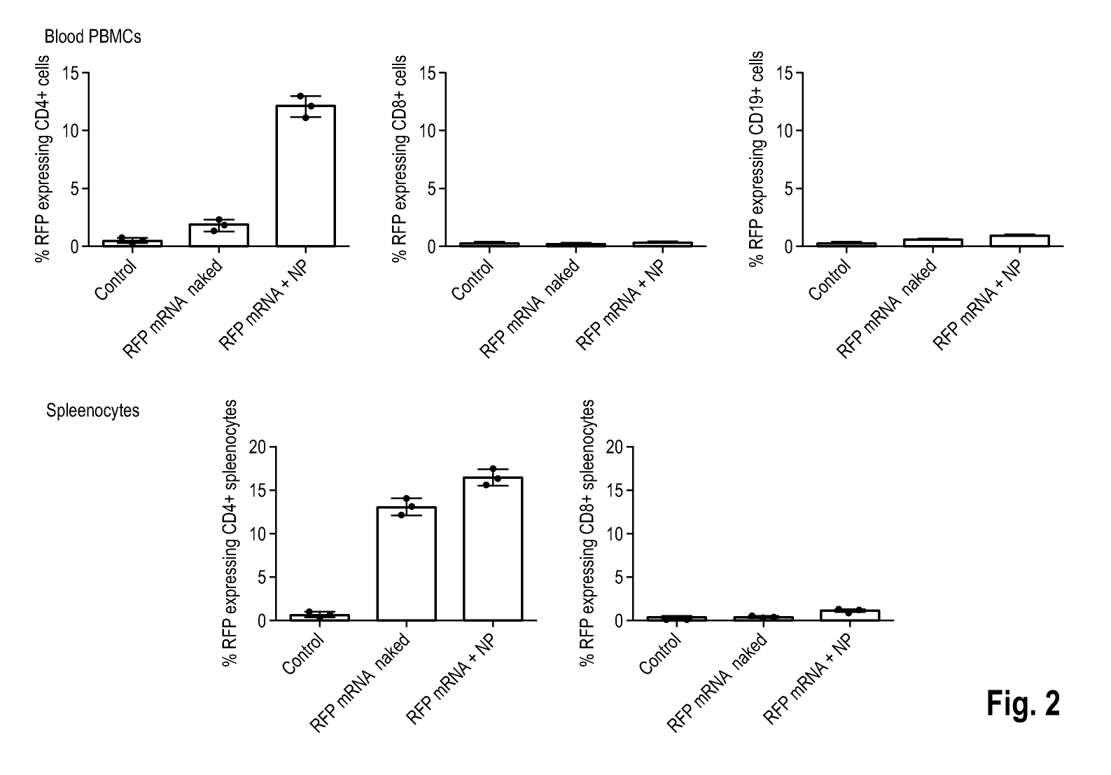

20 μg modified RFP-mRNA-NPs in a total volume of 100 μl were administered i.v. into the tail vein of BALB/c mice. After 24 h blood was withdrawn via retro-orbital bleeding, mice were sacrificed and spleenocytes were isolated. Immune cells were analyzed for RFP expression by flow cytometry. The data is shown in FIG. 2: RFP+ cells were determined and quantified via flow cytometry. In some cell contexts RFP mRNA+NP was significantly higher expressed compared to RFP mRNA alone (n=3 mice per group). *P<0.05, ***P<0.001 (Mann-Whitney-U tests).

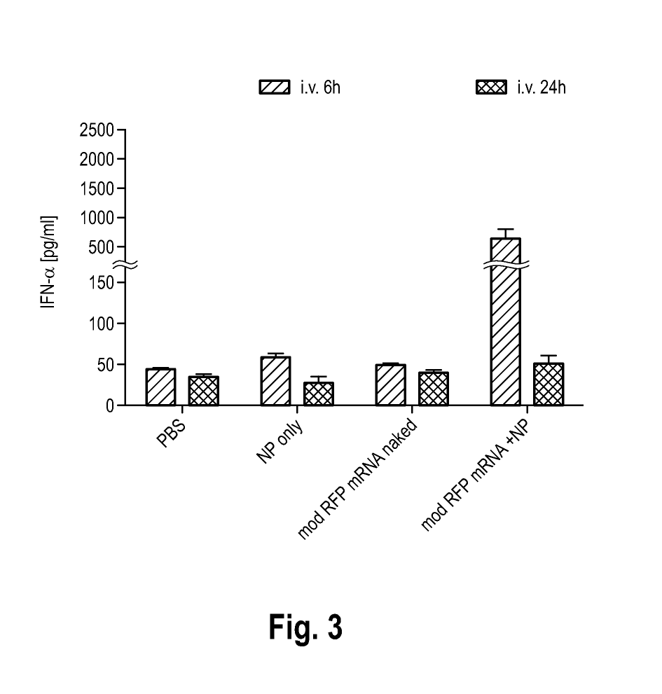

The immune reaction developed upon i.v. administration of RFP-mRNA-NPs was measured via quantification of IFN-alpha release after 6 h and 24 h using ELISA, as shown in FIG. 3: In vivo immune reaction to chemically modified RFP mRNA complex to NPs. 20 pg of RFP mRNA with or without NPs was i.v. injected into mice (n=3 mice per group). 6 h and 24 h post-injection, IFN-alpha was measured by ELISA in duplicates.

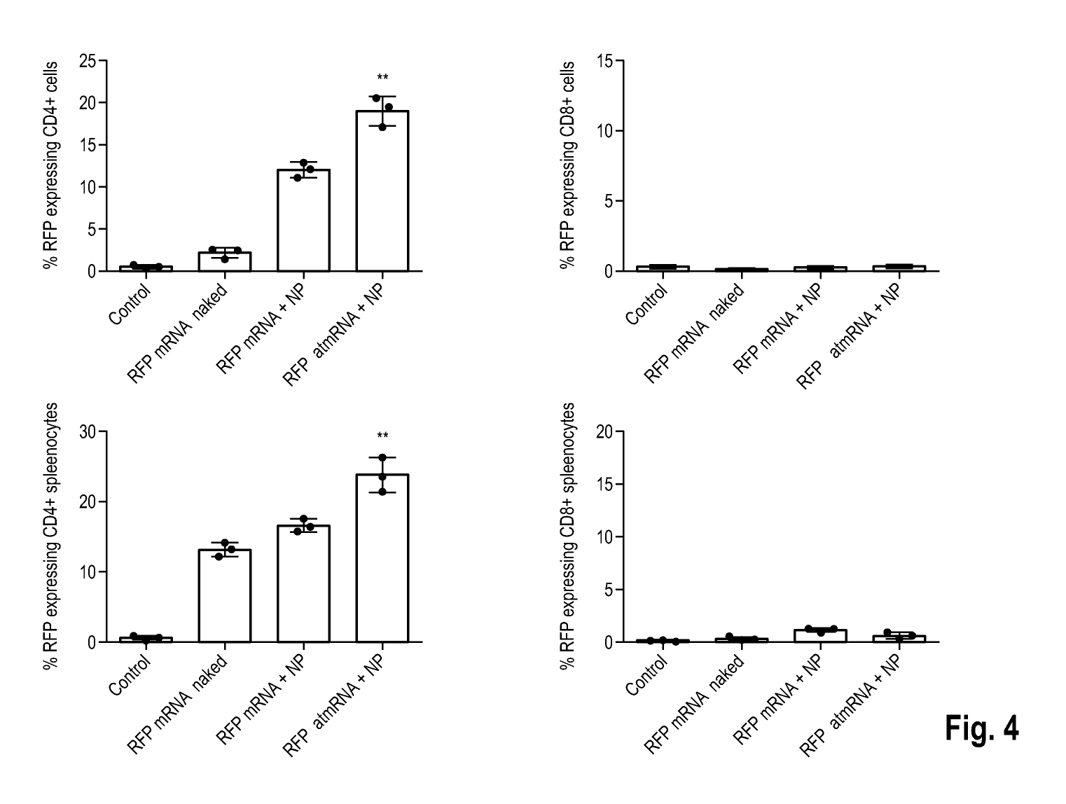

In a next experiment anti-CD4 aptamer sequence was added downstream of the polyA sequence. IVT was performed as described above to obtain atmRNA. i.v. application of the atmRNA to NSG mice, partially-humanized by i.v. injection of 25×10e6 human PBMCs two weeks prior to the start of the experiment, was performed as described above. After 24 h blood was withdrawn retroorbitally and immune cells were analyzed for RFP expression bei flow cytometry. The data is shown in FIG. 4: RFP+cells were determined and quantified via flow cytometry. In some cell contexts RFP atmRNA+NP was significantly higher expressed compared to regular RFP mRNA+NP (n=3 mice per group). **P<0.01 (Mann-Whitney-U tests).

Clearly, CD4+ T cells showed a significantly higher expression of RFP compared to the expression found when using chemically modified RFP mRNA without attached aptamer sequence.

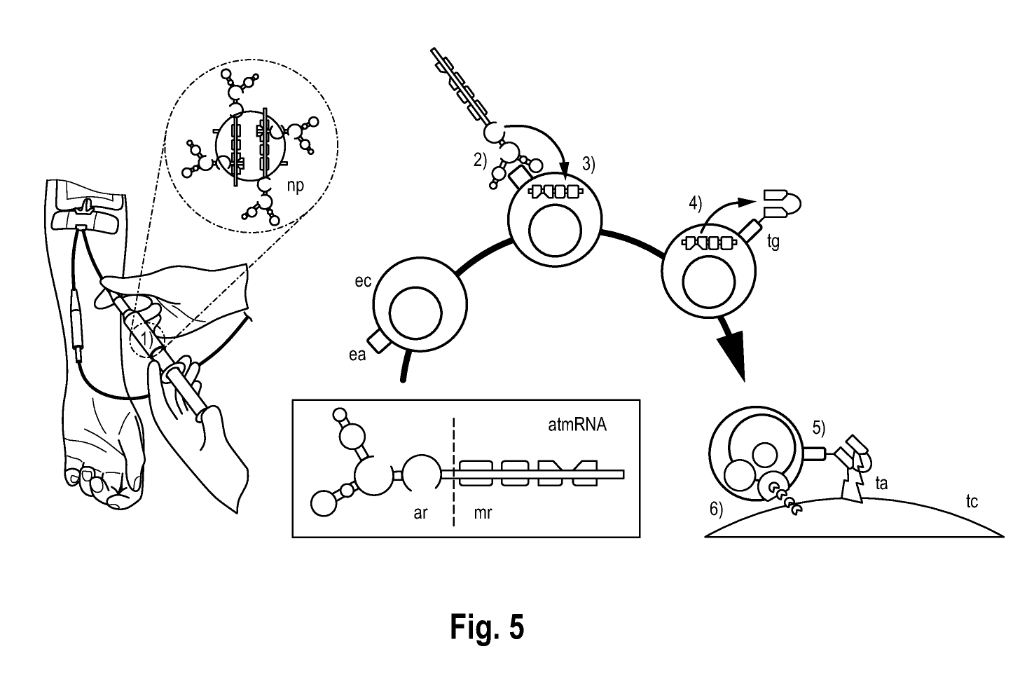

In a further experiment, RFP was substituted with a CD19-CD28-CD3-zeta construct, which—upon translation—assembles to an anti-CD19 CAR. The rationale behind that approach is depicted in FIG. 5: The aptamer-targeted modified messenger-RNA (atmRNA) consists of an aptamer (ar) targeting an effector antigen (ea) and a transgen (tg) (e.g. a chimeric antigen receptor, transgenic T cell receptor, transgenic T cell receptor with artificial costimulatory domain or any immunomodulatory receptor including reverse signaling and others) that is encoded by modified mRNA (mr). 1) atmRNA complexed with a nanoparticle (np) is injected intravenously. 2) atmRNA binds to the effector antigen (ea) and to an effector cell (ec) (e.g. CD4 on a T cell). 3) atmRNA bound on an effector antigen (ea) is internalized due to antigen flux. 4) transgen (tg) encoding modified messenger RNA gets translated in the cytosol of the target cell (tc) and the transgen (tg) gets expressed on the target cell (tg). 5) The transgen (e.g. a chimeric antigen receptor) binds to the target antigen (e.g. tumor associated antigen) on a target cell (tc) (e.g. tumor cell). 6) The effector cell (e.g. chimeric antigen expressing T cell) gets activated and mediates functions to the target cell (tc) (e.g. induces cell death in a tumor cell).

The DNA sequence encoding the CD19-CD28-CD3-zeta construct is as follows:

(SEQ ID No. 2)
5′-ATGCTTCTCCTGGTGACAAGCCTTCTGCTCTGTGAGTTACCACAC
CCAGCATTCCTCCTGATCCCAGACATCCAGATGACACAGACTACATCCTC
CCTGTCTGCCTCTCTGGGAGACAGAGTCACCATCAGTTGCAGGGCAAGTC
AGGACATTAGTAAATATTTAAATTGGTATCAGCAGAAACCAGATGGAACT
GTTAAACTCCTGATCTACCATACATCAAGATTACACTCAGGAGTCCCATC
AAGGTTCAGTGGCAGTGGGTCTGGAACAGATTATTCTCTCACCATTAGCA
ACCTGGAGCAAGAAGATATTGCCACTTACTTTTGCCAACAGGGTAATACG
CTTCCGTACACGTTCGGAGGGGGGACTAAGTTGGAAATAACAGGCTCCAC
CTCTGGATCCGGCAAGCCCGGATCTGGCGAGGGATCCACCAAGGGCGAGG
TGAAACTGCAGGAGTCAGGACCTGGCCTGGTGGCGCCCTCACAGAGCCTG
TCCGTCACATGCACTGTCTCAGGGGTCTCATTACCCGACTATGGTGTAAG
CTGGATTCGCCAGCCTCCACGAAAGGGTCTGGAGTGGCTGGGAGTAATAT
GGGGTAGTGAAACCACATACTATAATTCAGCTCTCAAATCCAGACTGACC
ATCATCAAGGACAACTCCAAGAGCCAAGTTTTCTTAAAAATGAACAGTCT
GCAAACTGATGACACAGCCATTTACTACTGTGCCAAACATTATTACTACG
GTGGTAGCTATGCTATGGACTACTGGGGTCAAGGAACCTCAGTCACCGTC
TCCTCAGTAGCAGATCCCGCCGAGCCCAAATCTCCTGACAAAACTCACAC
ATGCCCACCGTGCCCAGCACCTGAACTCCTGGGGGGACCGTCAGTCTTCC
TCTTCCCCCCAAAACCCAAGGACACCCTCATGATCTCCCGGACCCCTGAG
GTCACATGCGTGGTGGTGGACGTGAGCCACGAAGACCCTGAGGTCAAGTT
CAACTGGTACGTGGACGGCGTGGAGGTGCATAATGCCAAGACAAAGCCGC
GGGAGGAGCAGTACAACAGCACGTACCGGGTGGTCAGCGTCCTCACCGTC
CTGCACCAGGACTGGCTGAATGGCAAGGAGTACAAGTGCAAGGTCTCCAA
CAAAGCCCTCCCAGCCCCCATCGAGAAAACCATCTCCAAAGCCAAAGGGC
AGCCCCGAGAACCACAGGTGTACACCCTGCCCCCATCCCGGGATGAGCTG
ACCAAGAACCAGGTCAGCCTGACCTGCCTGGTCAAAGGCTTCTATCCCAG
CGACATCGCCGTGGAGTGGGAGAGCAATGGGCAGCCGGAGAACAACTACA
AGACCACGCCTCCCGTGCTGGACTCCGACGGCTCCTTCTTCCTCTACAGC
AAGCTCACCGTGGACAAGAGCAGGTGGCAGCAGGGGAACGTCTTCTCATG
CTCCGTGATGCATGAGGCTCTGCACAACCACTACACGCAGAAGAGCCTCT
CCCTGTCTCCGGGTAAAAAAGATCCCAAATTTTGGGTGCTGGTGGTGGTT
GGTGGAGTCCTGGCTTGCTATAGCTTGCTAGTAACAGTGGCCTTTATTAT
TTTCTGGGTGAGGAGTAAGAGGAGCAGGCTCCTGCACAGTGACTACATGA
ACATGACTCCCCGCCGCCCCGGGCCCACCCGCAAGCATTACCAGCCCTAT
GCCCCCCCACGCGACTTCGCAGCCTATCGCTCCCTGAGAGTGAAGTTCAG
CAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGA
CGTGGCCGGGACCCTGAGATGGGGGGAAAGCCGAGAAGGAAGAACCCTCA
GGAAGGCCTGTACAATGAACTGCAGAAAGATAAGATGGCGGAGGCCTACA
GTGAGATTGGGATGAAAGGCGAGCGCCGGAGGGGCAAGGGGCACGATGGC
CTTTACCAGGGTCTCAGTACAGCCACCAAGGACACCTACGACGCCCTTCA
CATGCAGGCCCTGCCCCCTCGCTAATCCTCGAGGGGAGACAAGAATAAAC
GCTCAATGACGTCCTTAGAATTGCGCATTCCTCACACAGGATCTTTTCGA
CAGGAGGCTCACAACAGGCTCCGGA-3′.

In the corresponding mRNA sequence of the anti-CD4 aptamer each “T” (thymine) is replaced by an “U” (uracil).

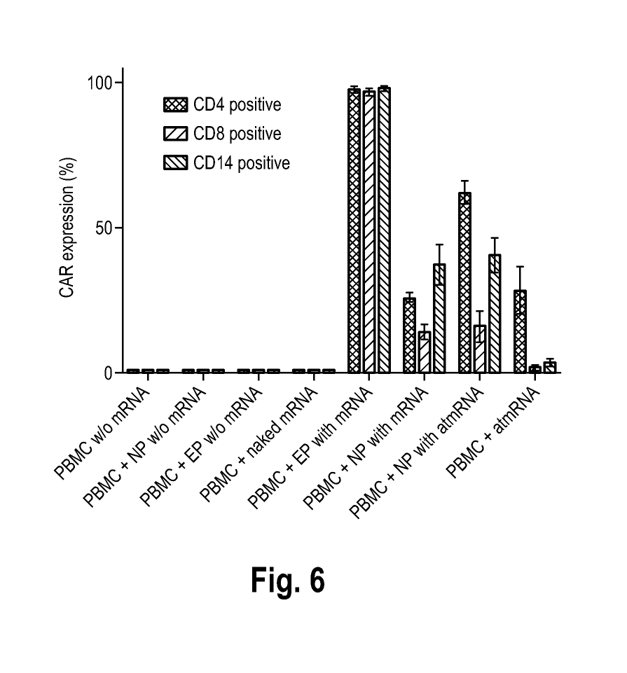

IVT of atmRNA encoding the anti-CD19 CAR was performed as described above. Freshly isolated human PBMCs were incubated for 24 h with 10 μg atmRNA targeted against CD4 with or without assembling to NP. mRNA encoding the anti-CD19 CAR with or without assembling to NP was used to demonstrate aptamer-specific expression. Electroporation of the same mRNA served as positive control. (n=4, technical replicates). The data is shown in FIG. 6: The experiment demonstrates a selective expression of the anti-CD19-CAR facilitated by atmRNA.

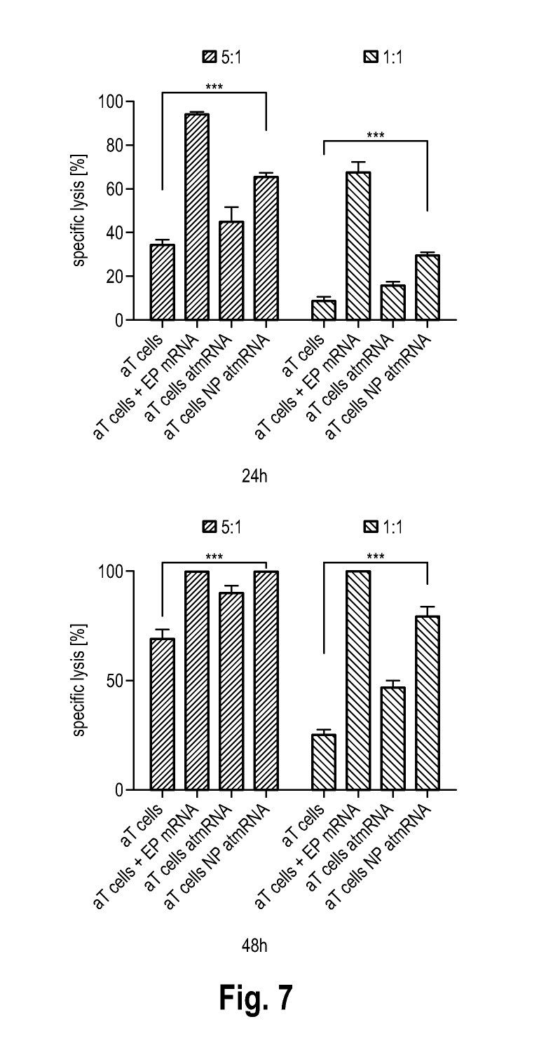

In the next experiment, activated T cells were pre-incubated for 24 h with 10 μg anti-CD19-CAR encoding atmRNA targeted against CD4 with or without assembling to NP. Pre-activation of CD4 and CD8-positive human T cells was performed by stimulation with anti-CD3/anti-CD28 activation beads and cultivation in IL-7 and IL-15 containing medium for 10 days. Electroporation of anti-CD19-CAR encoding mRNA served as positive control. After pre-incubation/electroporation, T cells were incubated with CD19 positive leukemic blasts (Nalm-6) at the effector to target ratios 5:1 and 1:1. Specific lysis was determined via bioluminescence (D-luciferin, Sigma Aldrich, www.sigmaaldrich.com) in a luciferase-based cytotoxicity assay using firefly luciferase constitutively expressing tumor cells. The data is shown in FIG. 7: atmRNA conditions showed an increased specific lysis of Nalm-6 CD19+ tumor cells after 24 and 48 h (n=6, technical replicates). ***P<0.001, activated T cells incubated with atmRNA assembled to NP (CAR19) vs. activated T cells (one-way ANOVA).

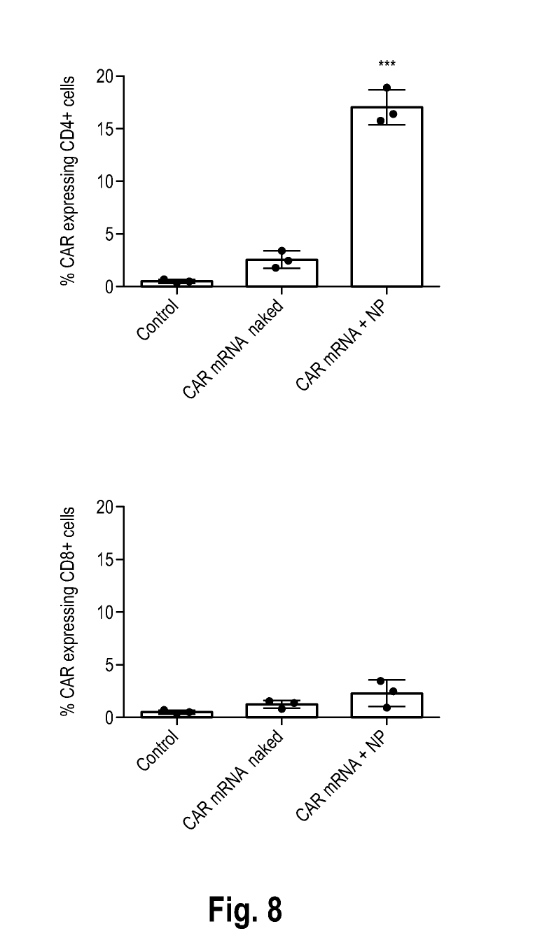

After in vitro evaluation, atmRNA was injected into the tail vein of NSG mice, partially humanized by previous i.v. injection of 25×10e6 human PBMCs two weeks prior to the start of the experiment. After 24 h, blood was withdrawn retroorbitally and immune cells were analyzed for CAR expression by flow cytometry. The data is shown in FIG. 8: CAR+ cells were determined and quantified via flow cytometry. In CD4+ cells, the CAR-construct using CAR atmRNA assembled to NP was significantly higher expressed compared to CAR atmRNA alone (n=4 mice per group). ***P<0.001, CAR mRNA+NP vs. CAR mRNA naked in CD4+ cells (Mann-Whitney-U tests).

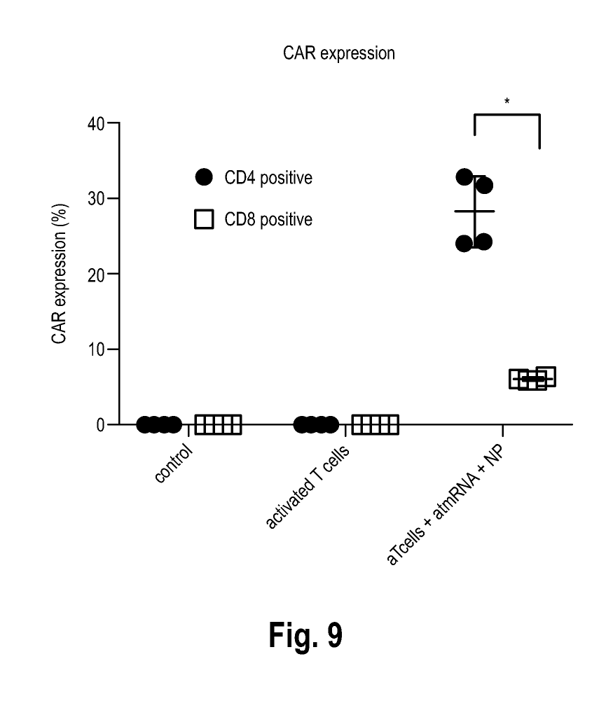

In the next experiment atmRNA was i.v. injected into leukemia baring NSG mice. NSG mice were injected i.v. with 1×10e6 CD19-positive Nalm-6 leukemic blasts. After 6 days, mice were transplanted with 2×10e7 pre-activated human T cells. One day after T cell application, atmRNA encoding anti-CD19-CAR assembled to nanoparticles was administered to the treatment group. After 48 h, blood was withdrawn retroorbitally and immune cells were analyzed for CAR expression by flow cytometry. The data is shown in FIG. 9. CAR+ cells were determined and quantified via flow cytometry. In CD4+ cells, injected with CAR atmRNA assembled to NP, CAR expression was significantly higher compared to CD8+ cells (n=4 mice per group). *P<0.05 (One-way ANOVA).

Functionality of expressed anti-CD19-CAR was further analyzed. In the above outlined experiment, mice were sacrificed 72 h after i.v. injection of CAR atmRNA assembled to NP. Bone marrow was analyzed for infiltration of leukemic blasts by flow cytometry using constitutive expression of mCherry on the Nalm-6 cell line for detection of blasts. Results demonstrate a significant reduction of blast infiltration in the bone marrow in the group activated T cells (aT cells)+atmRNA+NP compared to aT cells only (n=4 mice per group). *P<0.05 (One-way ANOVA). This effect has to be attributed to functional anti-CD19-CAR expression and specific T cell activation.

The data conclusively demonstrates that highly functional immunoreceptors, such as used in the above mentioned experiment, e.g. CARs, can be selectively expressed on specific immune cells such as CD4+ T cells. Using atmRNA naked or assembled to nanoparticles or liposomes, i.v. application is made possible, which overcomes all complicated and time-consuming ex vivo steps currently state-of-the-art to reprogram T cells. Thus, with the presented invention the genetic engineering of “designer T cells” can be done ultra-quick and “off-the-shelf” and opens up a vast range of possibilities to initiate/elicit antigen specific immune responses against cancer, infectious and immunologically triggered diseases.

4. Illustration of the Modular Design of the Product According to the Invention Exemplified by an Immune Receptor Expressing atmRNA

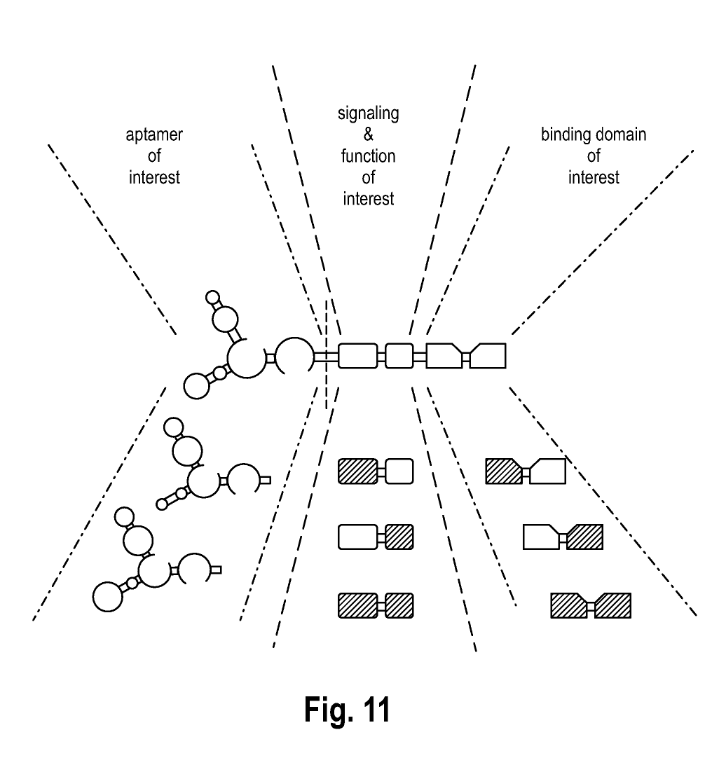

Reference is made to FIG. 11. The atmRNA-based immunoreceptor qualifies for targeting any cellular molecule expressed on the cell surface and internalized by any cell of interest via modular exchange of the aptamer specificity of interest (e.g. CD4, CD8, CD28, CD137 just to name a few, not excluding any others). Moreover the signaling and thereby the defined function is also based on a modular synthesis of predefined features and allows any available combinatory artificial signaling (e.g. activatory signaling [CD3ζ chain, CD28, CD137, OX40 just to name a few, not excluding any others], inhibitory signaling [PD-receptor, FAS-receptor just to name a few, not excluding any others], modulatory signaling [insulin and NFκB signaling just to name a few, not excluding any others]. The modular exchange of the binding domain (e.g. an scFv) by the target of interest facilitates the primary targeting and thus the fundamental on and off modulation of the downstream signaling by any predefined epitope structures, that are possibly targetable by a specific binding domain.

5. Illustration of Different Strategy how Immune Receptors, Expressed by atmRNA, can Modulate Effector as well as Target Cell Function

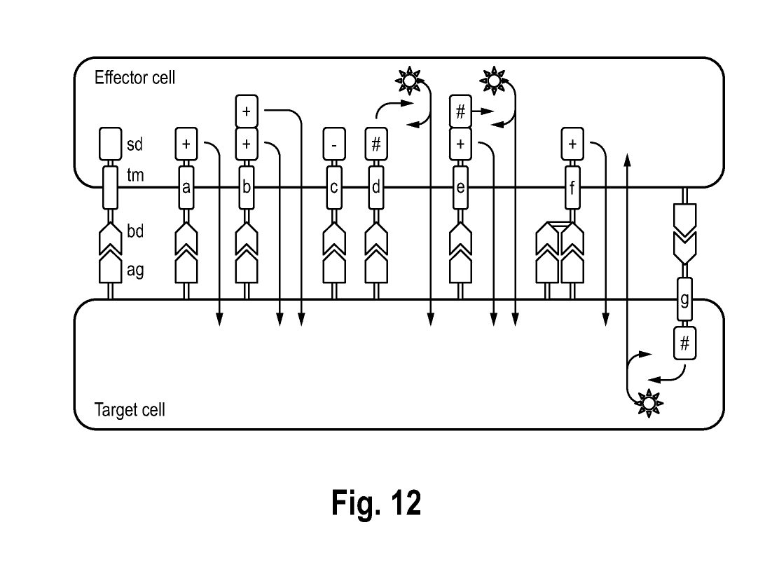

Reference is now made to FIG. 12. Immune receptors (IR) are composed of 1) an extracellular binding domain (bd) recognizing a specific antigen, 2) a transmembrane domain (td), 3) one or several signaling domains (sd). IR can mediate different functions upon specific ag recognition: a) activation of effector cell (EC) via an activating sd mediating effector function (→), e.g. induction of cell death, to a target cell (TC); b) enhanced activation of EC and enhanced effector function on TC by multiple sd; c) inhibition of EC via inhibitory sd; d) induction of specific gene expression in EC, mediating effect on EC and/or TC; e) activation of EC mediating effector function on TC and specific gene expression in EC mediating effect on EC and/or TC; f) simultaneous recognition of several ag using multiple bd; g) expression of IR on TC mediating e.g. specific gene expression in TC effecting TC and/or EC.

6. Example Sequence of Novel Modular Design Immune Receptor atmRNA

An example DNA sequence of a novel modular design immune receptor atmRNA is illustrated in the following.

Anti-CD19-41-BB-CD3ζ construct plus poly-a tail and sticky bridge:

(SEQ ID No. 3)
5′-
GCTAGCGCCGCCACC

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

GAATTCgagcagaagctgatctccgaagaggacctgACCACAACACC
CGCTCCTAGAC-CTCCAACACCAGCTCCAACAATCGCCAGCCAGCCTCTG
TCTCTCAGACCTGAGGCTTGTAGACCTGCTGCTGGCGGAGCCGTGCATAC
AAGAGGACTGGATTTCGCCTGCGACATCTACATCTGGGCTCCTCTGGCTG
GCACATGTGGCGTGCTGCTGCTGAGCCTGGTCATCACCCTGTATTGCAAG
CGGGGCAGAAAGAAACTGCTCTACATCTTCAAGCAGCCCTTCATGCGGCC
CGTGCAGACCACACAAGAGGAAGATGGCTGCTCCTGCAGATTCCCCGAGG
AAGAAGAAGGCGGCTGCGAGCTGAGAGTGAAGTTCAGCAGATCCGCCGAC
GCTCCTGCCTATCAGCAGGGCCAAAACCAGCTGTACAACGAGCTGAACCT
GGGGAGAAGAGAAGAGTACGACGTGCTGGACAAGCGGAGAGGCAGAGATC
CTGAAATGGGCGGCAAGCCCAGACGGAAGAATCCTCAAGAGGGCCTGTAT
AATGAGCTGCAGAAAGACAAGATGGCCGAGGCCTACAGCGAGATCGGAAT
GAAGGGCGAGCGCAGAAGAGGCAAGGGACACGATGGACTGTACCAGGGCC
TGAGCACCGCCACCAAGGATACCTATGATGCCCTGCACATGCAGGCCCTG
CCTCCAAGATAGAAGCTTCTCGA-Gaaaaaaaaaaaaaaaaaaaaaaaaa
aaaaaaaaaaaaaaaaaaaaaaaaaaaaaaaaaaaaaaaaaaaaaaaaaa
aaaaaaaaaaaaaaaaaaaaaaaaaaaaaaaaaaaaaaaaaaaaaGTCGA
CCTCCTAG-GAGCTCGGGCCC-3′ .

CD4 aptamer short stick:

(SEQ ID No. 4)
5′-
GAGGATCCTCGAGCCCGGTTTTTTTT [symbol] [symbol] [symbol] [symbol]
-3′

CD4 aptamer long stick:

(SEQ ID No. 5)
5′-
TTTTTTTTCAGCTGGAGGATCCTCGAGCCCGGTTTTTTTTGTGACGTCCT
GATCGATTGTGCATTCGGTGTGACGATCT-3′

CD8 aptamer short stick:

(SEQ ID No. 6)
5′-
GAGGATCCTCGAGCCCGGTTTTTTTT [symbol] [symbol] [symbol]
-3′

CD8 aptamer long stick:

(SEQ ID No. 7)
5′
TTTTTTTTCAGCTGGAGGATCCTCGAGCCCGGTTTTTTTT [symbol] [symbol] [symbol]
-3′

The bold italic capital letters illustrate the BINDING DOMAIN (anti CD19 scFv). The restriction side is shown in underlined normal small letters. The myc-tag is shown in normal small letters. The (CO)SIGNALING DOMAIN (41-BB-CD3ζ) is shown in normal italic capital letters. The bold small letters show the poly-a-tail. The bold capital letters show the STICKY BRIDGE. The SPACER is shown in normal capital letters. The APTAMER is shown in bold italic large underlined letters.

In the corresponding mRNA sequences each “T” (thymine) is replaced by an “U” (uracil).

### Sequences

SEQ ID no. 1: DNA sequence encoding the anti-CD4 aptamer;
SEQ ID no. 2: DNA sequence encoding the CD19-CD28-CD3-zeta construct
SEQ ID no. 3: DNA sequence encoding anti-CD19-41-BB-CD3ζ construct plus poly-a tail and sticky bridge
SEQ ID no. 4: DNA sequence encoding CD4 aptamer short stick
SEQ ID no. 5: DNA sequence encoding CD4 aptamer long stick
SEQ ID no. 6: DNA sequence encoding CD8 aptamer short stick
SEQ ID no. 7: DNA sequence encoding CD8 aptamer long stick

What is claimed is:

1. A product for an in vivo expression of a protein in a living being, comprising
a first entity comprising a first nucleic acid encoding an intracellularly expressible protein, and, associated therewith,
a second entity configured for a specific binding to a cellular structure of said living being.

2. The product of claim 1, wherein said first nucleic acid is an mRNA.

3. The product of claim 2, wherein said mRNA is a nucleotide-modified mRNA.

4. The product of claim 1, wherein said intracellularly expressible protein is a protein capable of effecting at least one phenomenon in said living being in a targeted manner, said phenomenon is selected from the group consisting of: immune response, cytokine expression, cell death induction, cell death inhibition, transcription factor expression, genetic modification, epigenetic modification.

5. The product of claim 4, wherein said intracellularly expressible protein is an antigen-specific receptor.

6. The product of claim 5, wherein said antigen-specific receptor is selected from the group consisting of:
T-cell receptor, tumor antigen-specific T-cell receptor, virus antigen specific T-cell receptor, bacterium antigen-specific T-cell receptor, fungus antigen-specific T-cell receptor, protozoan antigen-specific T-cell receptor;
chimeric antigen receptor (CAR), CAR targeting a tumor-associated antigen selected from the group consisting of: HER2/neu, ErbB, EGFR, EGFRvIII, FGFR3, FGFR4, LI-13R, II-13Rα2, II-11Rα, VEGFR2, ALK, GD2, GD3, mesothelin, Survivin, PMSA, PSCA, CEA, MUC1, GPC3, GPCS, CSPG4, ROR1, FR-α, FR-β, Igk, Lewis^{Y}, Glypican3, EphA2, CAIX, AFP, FAP, c-MET, HLA-DR, CA-125, CS1, BCMA, NKG2D ligands (MICA/MICB), CLL1, TALLA, LGR5, PD-L1, PD-L2, CD10, CD11b, CD14, CD15, CD19, CD20, CD22, CD29, CD30, CD32, CD33, CD34, CD38, CD44, CD44v6, CD44v7/8, CD45, CD47, CD56, CD64, CD66, CD79a, CD79b, CD95, CD99, CD112, CD117, CD123, CD133, CD135, CD138, CD146, CD152, CD157, CD171CD184, CD200, CD221, CD243, CD262, CD276, CD300f, CD305, CD326, CD338, CD366; CAR targeting a bacterium specific antigen, CAR targeting a fungus specific antigen, CAR targeting a virus specific antigen, CAR targeting a protozoan specific antigen.

7. The product of claim 4, wherein said intracellularly expressible protein is
a cell death inducing or inhibiting protein selected from the group consisting of: caspase, second mitochondria-derived activator of caspases (SMAC), BCL-2 family protein, inhibitor of apoptosis protein (IAP), tumor necrosis factor receptor superfamily (TNFRSF) protein, death-inducing signaling complex, p53, interferons, or
an immune modulatory protein selected from the group consisting of: cytokines, chemokines, tumor necrosis factor (TNF) family proteins and colony stimulating factors, or
a gene expression or protein modulating cellular signaling molecule selected from a group consisting of: kinases, phosphatases, acetyltransferases, deacetylases, methyltransferases, SUMOylating enzymes, and deSUMOylating enzymes, or
a gene sequence modulating molecule selected from the group consisting of: zinc-finger nucleases, meganucleases, TAL effector nucleases, CRISPR/Cas9 related nucleases, nickases, and FokI based dCas9 nucleases.

8. The product of claim 1, wherein said second entity is configured for a specific binding to a cell surface expressed protein characterizing a cell of the human hematopoiesis or a cell of the human immune system or both.

9. The product of claim 8, wherein said cell surface expressed protein is a cluster of differentiation (CD) protein or equivalent.

10. The product of claim 9, wherein said CD protein is selected from the group consisting of: CD4, CD8, CD3, CD10, CD16, CD19, CD20, CD22, CD25, CD28, CD30, CD33, CD34, CD38, CD44, CD44v6, CD44v7/8, CD45, CD45RA, CD45RO, CD56, CD62L, CD95, CD123, CD127, CD133, CD135, CD137, CD138, CD152, CD171, and wherein said equivalent is selected from the group consisting of: CCR4, CCR5, CCR6, CCR7, CXCR3, CXCR4, CXCR5, TCRαβ, TCRγδ, CTLA-4, PD1, TIM3, NKG2D, HER2/neu, ErbB, EGFR, EGFRvIII, FGFR3, FGFR4, LI-13R, II-13Rα2, II-11Rα, VEGFR2, ALK, GD2, GD3, mesothelin, survivin, PMSA, PSCA, CEA, MUC1, GPC3, GPC5, CSPG4, ROR1, FR-α, FR-β, Igk, Lewis^{Y}, glypican3, EphA2, CAIX, CSPG4, AFP, FAP, c-MET, HLA-DR, CA-125, CS1, BCMA, NKG2D ligands (MICA/MICB), PD1, PD-L1, PD-L2, CLL1, TALLA, LGR5.

11. The product of claim 1, wherein said second entity is an aptamer.

12. The product of claim 11, wherein said aptamer is an RNA aptamer.

13. The product of claim 11, wherein said aptamer is connected to said first nucleic acid by the concatenation of nucleotides resulting in a single-stranded nucleic acid molecule.

14. The product of claim 13, wherein the single stranded nucleic acid molecule is a single-stranded mRNA molecule.

15. The product of claim 11, wherein said aptamer is connected to said nucleic acid by the hybridization of complementary bases resulting in a double-stranded nucleic acid molecule.

16. The product of claim 15, wherein said double stranded nucleic acid molecule is a double-stranded mRNA molecule.

17. The product of claim 1, further comprising nanoparticles complexed with said first or said second entity or both.

18. The product of claim 1, further comprising liposomes packaging said first or said second entity or both.

19. A medicament comprising the product of claim 1 and a pharmaceutically acceptable carrier.

20. The method for the treatment a disease comprising the administration of the product of claim 1 or the medicament of claim 19 to a patient in need.

21. The method of claim 20, wherein said disease is selected from the group consisting of: a tumor and/or oncologic disease, a hematologic disease, an infectious disease, a rheumatologic disease, a genetic/hereditary disease, an autoimmune disease, an allergic disease.

22. A nucleotide-modified mRNA for an in vivo expression of a protein in a living being comprising
a first ribonucleotide sequence encoding an intracellularly expressible protein, and
a second ribonucleotide sequence encoding an aptamer configured for a specific binding to a cellular structure of said living being.


SEQUENCE LISTING (7 sequences)

SEQ ID NO: 1 - DNA, 86 bp, Artificial Sequence
  Anti-CD Aptamer
gggagacaagaataaacgctcaatgacgtccttagaattgcgcattcctcacacaggatcttttcgacaggaggctcacaacaggc

SEQ ID NO: 2 - DNA, 2170 bp, Artificial Sequence
  CD19-CD28-CD3-zeta consruct CDCce
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagtagcagatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgcccccccacgcgacttcgcagcctatcgctccctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaatcctcgaggggagacaagaataaacgctcaatgacgtccttagaattgcgcattcctcacacaggatcttttcgacaggaggctcacaacaggctccgga

SEQ ID NO: 3 - DNA, 1693 bp, Artificial Sequence
  Anti-CD19-41-BB-CD3-zeta Construct Plus Poly-A Tail and Sticky Bridge
gctagcgccgccaccatgttgctgctggttacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgaccatctccaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtttcttctgtggccgaccaagaattcgagcagaagctgatctccgaagaggacctgaccacaacacccgctcctagacctccaacaccagctccaacaatcgccagccagcctctgtctctcagacctgaggcttgtagacctgctgctggcggagccgtgcatacaagaggactggatttcgcctgcgacatctacatctgggctcctctggctggcacatgtggcgtgctgctgctgagcctggtcatcaccctgtattgcaagcggggcagaaagaaactgctctacatcttcaagcagcccttcatgcggcccgtgcagaccacacaagaggaagatggctgctcctgcagattccccgaggaagaagaaggcggctgcgagctgagagtgaagttcagcagatccgccgacgctcctgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagatagaagcttctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaagtcgacctcctaggagctcgggccc

SEQ ID NO: 4 - DNA, 65 bp, Artificial Sequence
  CD4 Aptamer Short Stick
gaggatcctcgagcccggttttttttgtgacgtcctgatcgattgtgcattcggtgtgacgatct

SEQ ID NO: 5 - DNA, 79 bp, Artificial Sequence
  CD4 Aptamer Long Stick
ttttttttcagctggaggatcctcgagcccggttttttttgtgacgtcctgatcgattgtgcattcggtgtgacgatct

SEQ ID NO: 6 - DNA, 55 bp, Artificial Sequence
  CD8 Aptamer Short Stick
gaggatcctcgagcccggttttttttctacagcttgctatgctccccttggggta

SEQ ID NO: 7 - DNA, 69 bp, Artificial Sequence
  CD8 Aptamer Long Stick
ttttttttcagctggaggatcctcgagcccggttttttttctacagcttgctatgctccccttggggta